(19) United States
(12) Patent Application Publication
(10) Pub. No.: US 2026/0242859 A1
(43) Pub. Date: Aug. 20, 2026

(54) FREEZE DRIED NUCLEIC ACID AMPLIFICATION MIXTURE

(71) Applicant: QIAGEN GmbH, Hilden (DE)

(72) Inventors: Miroslav VRANES, Hilden (DE); Stefan Otto CORNELIUS, Hilden (DE); Ralf PEIST, Hilden (DE); Thorsten SINGER, Hilden (DE)

(21) Appl. No.: 19/106,692

(22) PCT Filed: Aug. 31, 2023
(86) PCT No.: PCT/EP2023/073899

§ 371 Date: Feb. 26, 2025

(30) Foreign Application Priority Data
Aug. 31, 2022 (EP) 22193106.6

(51) Int. Cl. C12Q 1/686; C12Q 1/48; C12Q 1/6806; C12Q 1/6813; C12Q 1/6876

(52) U.S. Cl. CPC C12Q 1/686; C12Q 1/48; C12Q 1/6806; C12Q 1/6813; C12Q 1/6876; C12Y 207/07

(57) ABSTRACT

The present invention relates to a lyophilized nucleic acid amplification composition comprising, i) a first reagent mixture for performing a nucleic acid amplification or elongation reaction, and ii) an amplification/elongation adjuvant preferably selected from the group of sucrose, sorbitol, methylglucosides, glycine, arginine, proline, erythritol, xylitol, maltose, lactose, Ficoll, mannitol, dextran, and trehalose. The invention also relates to method of amplifying a nucleic acid sample comprising the steps of, a) providing a lyophilized first mixture composition comprising, i) reagents for performing a nucleic acid amplification reaction, ii) and an amplification adjuvant selected from the group of sucrose, sorbitol, methylglucosides, glycine, arginine, proline, erythritol, xylitol, maltose, lactose, Ficoll, mannitol, dextran and trehalose, b) re-hydrating the lyophilization cakes, c) optionally adding one or more oligonucleotide primers depending on whether or not these are present in the first mixture, d) adding a sample, and e) performing an amplification reaction.

### FIELD OF THE INVENTION

The present invention relates to the field of nucleic acid amplification/elongation, more particularly to the use of amplification/elongation adjuvants in freeze-dried nucleic acid amplification mixtures for use in nucleic acid amplification/elongation reactions.

### BACKGROUND

DNA polymerase chain reaction (hereinafter referred to as “PCR”) allows the DNA sequence at a specific region of a genome to be amplified by more than a million-fold, provided that at least part of its nucleotide sequence is already known. Various kinds of amplification techniques exist today.

In PCR sections of the sequence that surround the region to be amplified are used to design two synthetic DNA oligonucleotides, each complementary to one strand of the DNA double helix, respectively. These oligonucleotides serve as primers for in vitro DNA synthesis, which is catalyzed by a DNA polymerase, and they determine the ends of the final DNA fragment that is obtained. Each cycle of the PCR requires denaturation to separate two strands of the DNA double helix, annealing for specific hybridization to complementary DNA sequences, and extension for synthesis of DNA. For effective amplification, 30 to 40 cycles of reaction are required.

In conclusion, PCR by which a specific nucleotide sequence can be amplified in vitro from the genomic DNA, enables detection of a specific DNA sequence and acquisition of the DNA fragment of interest in large quantity in a short period of time. Other techniques allow also amplification of RNA.

The PCR technique has been utilized in a wide range of life sciences, such as in detection of genes associated with genetic diseases (see: Suzuki, Y. et al., Anal. Biochem., 192:82-84 (1991); Gibbs, R. A. et al., Nucleic Acids Res., 17:2374-2448 (1989); Ballabio, A. et al., Nature, 343:220 (1990)); in detection and expression of a specific mRNA by way of cDNA amplification by employing reverse transcription-PCR (RT-PCR) and RACE (rapid amplification of cDNA end) methods (see: Rappolee, D. A. et al., Science, 241:708-712 (1991); Frohman, M. A. et al., Proc. Natl. Acad. Sci., U.S.A., 85:8998-9002 (1988)); in Direct Amplification and Sequencing of DNA DEXAS (see Biol Chem, 1997 February; 378 (2): 99-105, Direct exponential amplification and sequencing (DEXAS) of genomic DNA-C. Kilger and S Pääbo, or Direct DNA sequence determination from total genomic DNA, Kilger C, Pääbo S.Nucleic Acids Res. 1997 May 15; 25 (10): 2032-4. doi: 10.1093/nar/25.10.2032) and for example real-time PCR.

In addition, it has been also utilized in diagnosis of a variety of diseases, such as HTLV-I and HBV (hepatitis B virus). It is likely the most important diagnostic tool and was recently used extensively during the CORONA pandemic.

Furthermore, applications of said PCR technique, e.g., DD-PCR (differential display-PCR), real-time PCR and Immuno-PCR have been developed, which permit detection of only a small portion of RNA or DNA in question which are not detectable using currently available methods.

In amplification of nucleic acid by PCR technique, every component of the reaction mixture for PCR, i.e., a template DNA, primers, reaction buffer, MgCl₂, KCl, but also PCR salts in general, dNTPs (dATP, dCTP, dGTP and dTTP) and DNA polymerase, must be mixed in step-wise fashion or simultaneously at the initial step, prior to initiating the reaction. Accordingly, it has been cumbersome to add and mix trace amounts of each component in a separate manner for every test sample, so experimental errors have frequently occurred. Especially when numerous samples are to be analyzed in a short period of time, inefficiency and occurring experimental errors have become serious obstacles in the experiments.

Moreover, it has been also known that aerosol formation when sample loading buffer is added to the PCR product, frequently causes carry-over contamination and leads to a false positive response, which has been an important problem to be solved, especially when used in diagnosis of diseases.

Lyophilized enzymes and PCR reagents have been known since about 1992 (Colaco, C., Sen, S., Thangavelu, M., Pinder, S., and Roser, B., Extradordinary Stability of Enzymes Dried in Trehalose: Simplified Molecular Biology, Bio/Technology 10:1007-1011, 1992 and U.S. Pat. No. 5,861,251A).

EP 0 917 568 A1 discloses various excipients but has not identified suitable concentrations and most importantly suitable applications. It relates primarily to the use in freezing enzymes without loss of activity.

However, it has been a challenge to maintain sensitivity levels reaching those of non-lyophilized reagent mixtures. In particular, in sensitive set-ups like real-time PCR. The present invention solves this problem now. In such applications a shift towards lower annealing temperatures would be helpful as well. Gelatine has previously been suggested in freeze-dried compositions as possible carrier protein in complex multi-bead assay systems (see US2006/0066399) and in particular to enhance the effects of anti-freeze proteins (see WO2005/076908). The present invention is not straightforward and was not predictable, substances as DMSO, betain or glycerin for example are not usable in a lyophilized cake.

### SUMMARY OF THE INVENTION

The present invention relates to a lyophilized nucleic acid amplification composition comprising,
a. a first reagent mixture for performing a nucleic acid amplification or elongation reaction,
b. an amplification/elongation adjuvant preferably selected from the group of sucrose, sorbitol, methylglucosides, glycine, arginine, proline, erythritol, xylitol, maltose, lactose, dextran, ficoll, mannitol, and trehalose, or any combination thereof.

Claimed excipients in such a composition are also, histidine, isoleucine, leucine, lysine, methionine, phenylalanine, threonine, tryptophan, valine, alanine, asparagine, aspartic acid, glutamic acid, serine, cysteine, glutamine, tyrosine and selenocysteine, or any combination thereof, or any of the aforementioned in combination with any of the other excipients according to the invention.

The present invention relates to a lyophilized nucleic acid amplification composition comprising,
a. a first reagent mixture for performing a nucleic acid amplification or elongation reaction,
b. an amplification/elongation adjuvant preferably selected from the group of sucrose, sorbitol, methylglucosides, glycine, arginine, proline, erythritol, xylitol, maltose, lactose, dextran, ficoll, mannitol, and trehalose.

Claimed excipients in such a composition are also, histidine, isoleucine, leucine, lysine, methionine, phenylalanine, threonine, tryptophan, valine, alanine, asparagine, aspartic acid, glutamic acid, serine, cysteine, glutamine, tyrosine and selenocysteine.

The present invention allows raising the sample volume without increasing the reaction volume therefore increasing sensitivity. Compositions prepared in accordance with the invention are stable for prolonged periods, even in the absence of anti-freeze proteins. A single solid composition such as a bead or cake system may generally be prepared which is economical and easy-to-use. When a composition is freeze-dried in the presence of a glass-forming reagent (ii), it generally forms a “cake” type 3-dimensional structure. This structure is supported by inclusion of a suitable stabilizer (iii) for the cake structure, and so this is a further component of the mixture. However, the compositions of the invention may also be suitable for use in other assays or reactions, in particular those which rely upon the use of enzymes to effect the procedures such as nucleic acid sequencing reactions, other nucleic acid amplification reactions (including ligase chain reaction (LCR), strand displacement amplification (SDA), transcription-mediated amplification (TMA), loop-mediated isothermal amplification (LAMP), rolling circle DNA amplification, digital PCR (dPCR), multiplex ligation-dependent probe amplification (MLPA) and multiple displacement amplification.)

Herein, “excipients” and “adjuvants” are used interchangeably.

Objectives of the invention are multipurpose dry amplification reagent mixtures for polymerase chain reaction preserving their reaction activity under prolonged storage at temperatures above 0° C. and allowing to provide specific PCR, as well as PCR analysis technique.

The above-mentioned objectives are achieved as follows; dry amplification reagent mixture for amplification contains polymerase, deoxynucleotide triphosphates, buffers, water-soluble dye, adjuvants presented by sucrose, sorbitol, methylglucosides, proline, erythritol and/or trehalose. Adjuvants can also be presented by, or presented in any combination of the subsequent adjuvants: mannitol, glycine, xylitol, maltose, arginine, lactose, ficoll, dextran, histidine, isoleucine, leucine, lysine, methionine, phenylalanine, threonine, tryptophan, valine, asparagine, aspartic acid, glutamic acid, cysteine, glutamine, tyrosine, selenocysteine, serine, alanine, or any combination thereof.

In principle, all of the adjuvants according to the invention can be used as single adjuvant or in combination with any other of the aforementioned adjuvants. When adjuvants are combined, it usually makes sense to combine 2 to 10, more preferably 2 to 5 and most preferably 2 to 4 of the adjuvants. Thus, different objectives, such as the resistance of the lyophilized cake against air humidity or the influence on the melting temperature in PCR reactions can be modulated by choosing different adjuvants as described in this application. The combination of different adjuvants might require adjustments in the setting of the lyophilization protocols.

The inventors have also found that the present invention solves a particularly demanding problem. This problem is that in mixed DNA samples in particular mixed DNA samples from forensic applications often only trace amounts of DNA of interest are present which one would like to amplify. However, in order to do so one would need very large amounts of sample. The inventors have therefore for the first time developed an entirely new technique which involves using lyophilized PCR amplification mixtures for forensic samples in particular for forensic samples that comprise trace amounts of target nucleic acid. Thus, in a preferred embodiment the present invention relates to the use of the composition in the field of forensics in particular in the field of amplifying forensic samples with nucleic acid mixtures and in preferred embodiments of amplifying mixtures that comprise small amounts of nucleic acids without the need for changing PCR conditions. Such small amounts may be in the range of between one and 10 nucleic acid target molecules, one and 50 nucleic acid target molecules, or one and 200 nucleic acid target molecules. Such small amounts may also be in the range of between one and 9 nucleic acid target molecules. These ranges require often large volumes. As stated above this is not possible with standard PCR mixes.

In a very preferred embodiment, the invention is applied to digital PCR. The beneficial effect of the new excipients has been observed also here.

PCR-analysis technique includes dissolving of dry amplification reagent mixture (cake) for polymerase chain reaction in water or a buffer solution containing Mg ions with subsequent addition of specific primers if needed and analyte DNA sample.

Preferred primers have a length of about 15-100, more preferably about 20-50, most preferably about 20-40 bases.

Oligonucleotides and oligonucleotide probes may also be used. These are usually 15-100 nucleotides long, more preferably about 20-50, most preferably about 20-40 bases long.

The amplification methods will comprise buffers, dNTPs or NTPs in addition to the enzymes required.

As used herein, the term “dNTP” refers to deoxyribonucleoside triphosphates. Non-limiting examples of such dNTPs are dATP, dGTP, dCTP, dTTP, dUTP, which may also be present in the form of labelled derivatives, for instance comprising a fluorescent label, a radioactive label, a biotin label. dNTPs with modified nucleotide bases are also encompassed, wherein the nucleotide bases are for example hypoxanthine, xanthine, 7-methylguanine, inosine, xanthinosine, 7-methylguanosine, 5,6-dihydrouracil, 5-methylcytosine, pseudouridine, dihydrouridine, 5-methylcytidine.

As used herein, the term “NTP” refers to ribonucleoside triphosphates. Non-limiting examples of such NTPs are ATP, GTP, CTP, TTP, UTP, which may also be present in the form of labelled derivatives, for instance comprising a fluorescent label, a radioactive label, a biotin label.

Preferably, the amplification method is the polymerase chain reaction (PCR) method.

A PCR reaction may consist of 10 to 100 “cycles” of denaturation and synthesis of a DNA molecule. In a preferred embodiment, the temperature at which denaturation is done in a thermocycling amplification reaction is between about 90° C. to greater than 95° C., more preferably between 92° C.-95° C. Preferred thermocycling amplification methods include polymerase chain reactions involving from about 10 to about 100 cycles, more preferably from about 25 to about 50 cycles, and peak temperatures of from about 90° C. to greater than 95° C., more preferably 92° C.-94° C. In a preferred embodiment, a PCR reaction is usually done using a DNA Polymerase originating from a thermophilic prokaryote to produce the PCR product, in exponential quantities relative to the number of reaction steps involved, at least one target nucleic acid sequence, given (a) that the ends of the target sequence are known in sufficient detail that oligonucleotide primers can be synthesized which will hybridize to them and (b) that a small amount of the target sequence is available to initiate the chain reaction.

Prepared composition mixtures can be stored for a long time (one year and more) at the wide range of temperatures (from −20° C. to +30° C.) without loss of activity.

The suggested amplification mixture is compatible with any primer system for genomic DNA, plasmid DNA, mitochondrial DNA, recombinant (genetically modified) DNA and can be used for PCR analysis for DNA of any multicellular or unicellular organisms, food products or their components as well as for quantitative determination of DNA in the samples.

The invention also relates to a method of amplifying a nucleic acid sample comprising the steps of,
a. providing a lyophilized composition comprising,
   i. a first mixture for performing a nucleic acid amplification reaction,
   ii. and an amplification adjuvant selected from the group of sucrose, sorbitol, methylglucosides, glycine, arginine, proline, erythritol, xylitol, maltose, lactose, Ficoll, mannitol, dextran, and trehalose, or any combination thereof.
b. re-hydrating the lyophilization cakes,
c. optionally adding one or more oligonucleotide primers depending on whether or not these are present in the first mixture,
d. adding nucleic acid sample, and
e. performing an amplification reaction.

Claimed excipients in such a method are also, histidine, isoleucine, leucine, lysine, methionine, phenylalanine, threonine, tryptophan, valine, alanine, asparagine, aspartic acid, glutamic acid, serine, cysteine, glutamine, tyrosine and selenocysteine, or any combination thereof, or any of the aforementioned in combination with any of the other excipients according to the invention.

The invention also relates to a method of amplifying a nucleic acid sample comprising the steps of,
a. providing a lyophilized composition comprising,
   i. a first mixture for performing a nucleic acid amplification reaction,
   ii. and an amplification adjuvant selected from the group of sucrose, sorbitol, methylglucosides, glycine, arginine, proline, erythritol, xylitol, maltose, lactose, Ficoll, mannitol, dextran, and trehalose.
b. re-hydrating the lyophilization cakes,
c. optionally adding one or more oligonucleotide primers depending on whether or not these are present in the first mixture,
d. adding nucleic acid sample, and
e. performing an amplification reaction.

Claimed excipients in such a method are also, histidine, isoleucine, leucine, lysine, methionine, phenylalanine, threonine, tryptophan, valine, alanine, asparagine, aspartic acid, glutamic acid, serine, cysteine, glutamine, tyrosine and selenocysteine.

Likewise, the invention relates to a method of preparing a lyophilized nucleic acid amplification composition comprising the steps, mixing in liquid form of
a. a first reagent mixture for performing a nucleic acid amplification or elongation reaction,
b. an amplification/elongation adjuvant preferably selected from the group of sucrose, sorbitol, methylglucosides, glycine, arginine, proline, erythritol, xylitol, maltose, lactose, Ficoll, mannitol, dextran, and trehalose, or any combination thereof.

Claimed excipients in such a method are also, histidine, isoleucine, leucine, lysine, methionine, phenylalanine, threonine, tryptophan, valine, alanine, asparagine, aspartic acid, glutamic acid, serine, cysteine, glutamine, tyrosine and selenocysteine, or any combination thereof, or any of the aforementioned in combination with any of the other excipients according to the invention.

Likewise, the invention relates to a method of preparing a lyophilized nucleic acid amplification composition comprising the steps, mixing in liquid form of
a. a first reagent mixture for performing a nucleic acid amplification or elongation reaction,
b. an amplification/elongation adjuvant preferably selected from the group of sucrose, sorbitol, methylglucosides, glycine, arginine, proline, erythritol, xylitol, maltose, lactose, Ficoll, mannitol, dextran, and trehalose.

### DETAILED DESCRIPTION OF THE INVENTION

The inventors have solved the above-mentioned problems by the present invention.

The present invention relates to a lyophilized nucleic acid amplification composition comprising,
a. a first reagent mixture for performing a nucleic acid amplification or elongation reaction, and
b. an amplification/elongation adjuvant preferably selected from the group of sucrose, sorbitol, methylglucosides, glycine, arginine, proline, erythritol, xylitol, maltose, lactose, Ficoll, mannitol, dextran, and trehalose, or any combination thereof.

Claimed excipients in such a composition are also, histidine, isoleucine, leucine, lysine, methionine, phenylalanine, threonine, tryptophan, valine, alanine, asparagine, aspartic acid, glutamic acid, serine, cysteine, glutamine, tyrosine and selenocysteine, or any combination thereof, or any of the aforementioned in combination with any of the other excipients according to the invention.

The present invention relates to a lyophilized nucleic acid amplification composition comprising,
a. a first reagent mixture for performing a nucleic acid amplification or elongation reaction, and
b. an amplification/elongation adjuvant preferably selected from the group of sucrose, sorbitol, methylglucosides, glycine, arginine, proline, erythritol, xylitol, maltose, lactose, Ficoll, mannitol, dextran, and trehalose.

Claimed excipients in such a composition are also, histidine, isoleucine, leucine, lysine, methionine, phenylalanine, threonine, tryptophan, valine, alanine, asparagine, aspartic acid, glutamic acid, serine, cysteine, glutamine, tyrosine and selenocysteine.

More than one amplification/elongation adjuvant according to the invention may be present in the composition according to the invention. Hence, mixtures of two or more amplification/elongation adjuvants selected from the group of sucrose, sorbitol, methylglucosides, glycine, arginine, proline, erythritol, xylitol, maltose, lactose, Ficoll, mannitol, dextran, trehalose, histidine, isoleucine, leucine, lysine, methionine, phenylalanine, threonine, tryptophan, valine, alanine, asparagine, aspartic acid, glutamic acid, serine, cysteine, glutamine, tyrosine and selenocysteine are also encompassed. In such a case, it is preferred that the lyophilized composition comprises a combination of i) sucrose and mannitol, or ii) sucrose, mannitol, and xylitol, or iii) sucrose, trehalose, and glycine, or iv) trehalose and dextran; more preferably, a combination of i) 0.5-3% w/v sucrose and 4-16% w/v mannitol, or ii) 0.5-3% w/v sucrose, 4-16% w/v mannitol, and 0.5-2% w/v xylitol, or iii) 0.5-3% w/v sucrose, 1-3% w/v trehalose, and 1-5% w/v glycine, or iv) 2-10% w/v trehalose, and 1-6% w/v dextran; even more preferably, a combination of i) 2% w/v sucrose and 8% w/v mannitol, or ii) 1% w/v sucrose, 8% w/v mannitol, and 1% w/v xylitol, or iii) 1.5% w/v sucrose, 1.5% w/v trehalose, and 3% w/v glycine, or iv) 3-7% w/v trehalose (most preferably 6% trehalose), and 2-4% w/v dextran; most preferably, a combination of 1% w/v sucrose, 8% w/v mannitol, and 1% w/v xylitol.

The invention relates to the use of a lyophilized nucleic acid amplification composition comprising, (a) a first reagent mixture for performing a nucleic acid amplification or elongation reaction, and (b) an adjuvant preferably selected from the group of sucrose, sorbitol, methylglucosides, glycine, arginine, proline, erythritol, xylitol, maltose, lactose, Ficoll, mannitol, dextran, trehalose, alanine and serine, or any combination thereof, for amplifying a forensic sample with trace amounts of target nucleic acid of interest and/or a mixture of nucleic acids. Such small trace amounts may be in the range of between 1 and 9 nucleic acid target molecules, 1 and 10 nucleic acid target molecules, 1 and 50 nucleic acid target molecules, or 1 and 200 nucleic acid target molecules in the sample to be amplified. These ranges require often large volumes as a lot of sample needs to be dissolved. As stated above this is not possible with standard PCR which only allows a limited volume size. Hence, it is beneficial to have a larger sample volume in such cases. This has so far not been possible. The inventors have now found that by providing for an amplification cake including such preferred excipients as outlined here in the combination of amplification reaction mixture and excipient and large volumes makes it possible to amplify such difficult samples in small reaction volumes usually used.

The invention relates to the use of a lyophilized nucleic acid amplification composition comprising, (a) a first reagent mixture for performing a nucleic acid amplification or elongation reaction, and (b) an adjuvant preferably selected from the group of sucrose, sorbitol, methylglucosides, glycine, arginine, proline, erythritol, xylitol, maltose, lactose, Ficoll, mannitol, and trehalose, for amplifying a forensic sample with trace amounts of target nucleic acid of interest and/or a mixture of nucleic acids. Such small trace amounts may be in the range of between 1 and 10 nucleic acid target molecules, 1 and 50 nucleic acid target molecules, or 1 and 200 nucleic acid target molecules in the sample to be amplified. These ranges require often large volumes as a lot of sample needs to be dissolved. As stated above this is not possible with standard PCR which only allows a limited volume size. Hence, it is beneficial to have a larger sample volume in such cases. This has so far not been possible. The inventors have now found that by providing for an amplification cake including such preferred excipients as outlined here in the combination of amplification reaction mixture and excipient and large volumes makes it possible to amplify such difficult samples in small reaction volumes usually used.

Preferably in the composition according to the invention the first mixture comprises one or more oligonucleotide primer(s) needed for nucleic acid amplification or elongation, or in another embodiment these are absent in the reagent mixture and are added subsequently.

Oligonucleotide herein is a low molecular weight deoxyribo-, ribo-, copolymers of deoxyribo- and ribonucleic acids of chain lengths between 3 and 150. Such oligonucleotides can have modified nucleotide residues such as —O-methoxy, phosphorothio-, methylphosphonates and others known in art. Primers herein are oligonucleotides which are used for extension reaction by a nucleic acid polymerase after a template primer hybrid is formed. Such primers can carry sequences specific for transcription by an RNA polymerase. Nucleic acid probes herein are nucleic acids with substantially complementary sequences to the target nucleic acids for detection or capture from a mixture. Such probes can be labeled for detection or immobilized onto a solid support to enrich the target by capture. A probe can be a single stranded or partially double stranded and can be an oligonucleotide or a larger nucleic acid.

Probes may be added to the present reactions. Probes may be labelled just like primers herein. Such preferred labels are disclosed herein.

Suitably the set of reagents of the first mixture above further comprises a buffer, salt (such as optionally magnesium or manganese salts for example magnesium or manganese halides), one or more primers and nucleotides required to from the extended PCR products attached to the primer(s) which are required to affect a polymerase chain reaction to amplify a target DNA sequence. However, it is possible that one or more of these elements may be missing in particular where these elements can be readily added later, for example in a rehydration buffer used to reconstitute the dried composition ready for use. In particular, the necessary salts may be added in this way and so the set of reagents may omit the above salts. Where this is done, the composition may be supplied in the form of a kit with rehydration buffer, containing the necessary salt supplements.

The composition may further comprise a labelled oligonucleotide useful in monitoring the progress of a polymerase chain reaction in real time. Also as used herein, the expression “real-time” means that the polymerase chain reaction can be monitored as it progresses and without halting or opening the reaction vessel. By monitoring how the amplification occurs and in particular at which cycles exponential increase in amplicon becomes significant allows the amount of target nucleic acid present in the sample being subject to the PCR to be quantified as is well known and understood in the art.

In one embodiment, the adjuvant in the composition according to the invention is selected from the group of,
a. sucrose,
b. sorbitol,
c. methylglucosides,
d. trehalose,
e. erythritol,
f. mannitol,
g. glycine,
h. proline,
i. xylitol,
j. maltose,
k. arginine,
l. lactose,
m. Ficoll,
n. dextran,
o. histidine,
p. isoleucine,
q. leucine,
r. lysine,
s. methionine,
t. phenylalanine,
u. threonine,
v. tryptophan,
w. valine,
x. asparagine,
y. aspartic acid,
z. glutamic acid,
aa. cysteine,
bb. glutamine,
cc. tyrosine,
dd. selenocysteine,
ee. serine, and
ff. alanine,
or any combination thereof.

In one preferred embodiment, the adjuvant in the composition according to the invention is selected from the group of,
a. sucrose,
b. sorbitol,
c. methylglucosides,
d. trehalose,
e. erythritol,
f. mannitol,
g. glycine,
h. proline,
i. xylitol,
j. maltose,
k. dextran,
l. serine, and
m. alanine,
or any combination thereof.

Weight and volume of the lyophilized cake are difficult to determine. Hence, if not indicated otherwise, percentages for the adjuvants/excipients indicated in this application are expressed as % w/v, e.g. 18% w/v of sucrose in the liquid mixture to be lyophilized into a cake.

The expression “the concentration of the adjuvant in the lyophilized composition” relates to the concentration of the adjuvant in the liquid mixture to be lyophilized into a cake.

Preferably in the composition according to the invention, the concentration (w/v) of the adjuvant in the lyophilized composition are as follows,
a. sucrose 12 to 22%,
b. sorbitol 14 to 60%,
c. methylglucosides 10 to 40%,
d. trehalose 6 to 18%,
e. mannitol 2 to 10%,
f. dextran 1 to 4%,
g. glycine 2 to 15%,
h. maltose 6 to 22%,
i. xylitol 6 to 22%,
j. proline 4 to 18%,
k. erythritol 6 to 20%,
l. serine 2 to 30%, and
m. alanine 2 to 14%.

Preferably in the composition according to the invention, the concentration of the adjuvant in the lyophilized composition are as follows,
a. sucrose,
b. sorbitol,
c. methylglucosides,
d. trehalose,
e. glycine,
f. maltose,
g. xylitol,
h. proline, and
i. erythritol.

Preferably in the composition according to the invention, the preferred adjuvant is selected from the group of,
a. sucrose,
b. sorbitol,
c. methylglucosides,
d. trehalose,
e. dextran, and
f. erythritol.

These create very good cakes. Mannitol and glycine also create very good cakes and are thus also preferred.

Sucrose has been seen to have some advantages and is preferred in the composition.

Claimed excipients in such a composition are also amino acids, such as histidine, isoleucine, leucine, lysine, methionine, phenylalanine, threonine, tryptophan, valine, alanine, asparagine, aspartic acid, glutamic acid, serine, cysteine, glutamine, tyrosine and selenocysteine.

The best has been seen to be and are thus most preferred, sucrose, sorbitol and dextran, and most preferably, sucrose. Such a composition according to the invention is adjusted so that the final concentration in the amplification mixture to be cycled is 12% to 22% of sucrose, more preferably 13% to 19% of sucrose, and more preferably 16% to 19% of sucrose, more preferably even 17% to 19% of sucrose and finally and most preferably 18% of sucrose.

For applications in which a high resistance of the lyophilized cake against air humidity is sought, sucrose is used in combination with other excipients and the final concentration of sucrose in the amplification mixture is between 0.5% to 3%, or between 0.5% to 2%, or 2%, or 1.5%, or 1%, or 0.5%.

In an optional embodiment 0.5% to 4% of dextran (final conc. In amplification mixture) are added to compositions according to the invention, preferably 0.8% to 2% of dextran are added and even more preferably, about 1% of dextran is added.

In one embodiment, 14% w/v to 60% w/v of sorbitol (final conc. in amplification mixture) is added to compositions according to the invention, preferably 17% w/v to 19% w/v of sorbitol is added, even more preferably 18% w/v of sorbitol is added.

In one embodiment, 2% w/v to 30% w/v of serine (final conc. in amplification mixture) is added to compositions according to the invention, preferably 2% w/v to 20% w/v of serine is added, even more preferably 3% w/v to 10% w/v of serine is added, most preferably about 4% w/v of serine is added.

In one embodiment, 2% w/v to 14% w/v of alanine (final conc. in amplification mixture) is added to compositions according to the invention, preferably 2% w/v to 12% w/v of alanine is added, even more preferably 3% w/v to 10% w/v of alanine is added, most preferably about 4% w/v of alanine is added.

The invention also relates to a method of amplifying a nucleic acid sample comprising the steps of,
a. providing a lyophilized composition comprising,
   i. a first reagent mixture for performing a nucleic acid amplification reaction,
   ii. and an amplification adjuvant being sucrose,
b. re-hydrating the lyophilization cakes, with water, a buffer or sample,
c. optionally adding one or more oligonucleotide primers depending on whether these are present in the first reagent mixture,
d. adding a sample,
e. performing an amplification reaction,
f. wherein the concentration of sucrose in the final reaction is 12% to 22% of sucrose, more preferably 13% to 19% of sucrose, and more preferably 16% to 19% of sucrose, more preferably even 17% to 19% of sucrose and finally and most preferably 18% of sucrose, and
g. optionally and preferably, dextran is added at a concentration of 0.5% to 4% of dextran (final conc. In amplification mixture).

The dextran concentration is usually 0.5% to 4% of dextran (final conc. In amplification mixture) it may be preferably 0.8% to 2% of dextran and even more preferably, about 1% of dextran.

The invention also relates to a method of amplifying a nucleic acid sample comprising the steps of,
a. providing a lyophilized composition comprising,
   i. a first reagent mixture for performing a nucleic acid amplification reaction,
   ii. and an amplification adjuvant being sucrose,
b. re-hydrating the lyophilization cakes, with water, a buffer or sample,
c. optionally adding one or more oligonucleotide primers depending on whether these are present in the first reagent mixture,
d. adding a sample,
e. performing an amplification reaction,
f. wherein the concentration of sucrose in the final reaction is 12% w/v to 22% w/v of sucrose, more preferably 13% w/v to 19% w/v of sucrose, and more preferably 16% w/v to 19% w/v of sucrose, more preferably even 17% w/v to 19% w/v of sucrose and finally and most preferably 18% w/v of sucrose.

Some have been seen to have no influence on melting temperatures (tm). These are then preferred if needed, dextran, arginine, lactose, Ficoll, and mannitol.

Some have been seen to have the same tm influence as glycerin, such as sorbitol, sucrose, and methylglucosides, and erythrol, these are then preferred if needed.

### Tm Temperature and Annealing Temperature in PCR Reactions:

The melting temperature (Tm) of DNA or oligonucleotides is defined as the temperature at which half of the DNA strands are in the random coil/double stranded or single-stranded (ssDNA) state. Tm depends on the length of the DNA molecule and its specific nucleotide sequence. DNA, when in a state where its two strands are dissociated (i.e., the dsDNA molecule exists as two independent strands), is referred to as having been denatured by the high temperature. PCR amplification comprises different steps like DNA denaturation, annealing of primers and extension in order to amplify the sequence or sequences of interest. The crucial step in PCR is the annealing of primers, where the annealing temperature determines the specificity of primer annealing. The annealing temperature of a standard PCR protocol is typically e.g. 55° C. or 60° C. The chosen temperature depends on the strand-melting temperature of the primers and the desired specificity. For greater stringency higher temperatures are recommended. Specificity and stringency of the PCR can be further modulated by including additives like glycerol or betaine which have been described to modify the Tm temperature or reduce secondary structures at GC rich regions (Jensen M A, Fukushima M, Davis R W (2010). DMSO and Betaine Greatly Improve Amplification of GC-Rich Constructs in DeNovoSynthesis. PLOS ONE 5 (6):e11024; Cheng S, Fockler C, Barnes W M, Higuchi R. Effective amplification of long targets from cloned inserts and human genomic DNA. Proc Natl Acad Sci USA. 1994; 91 (12): 5695-5699. doi: 10.1073/pnas.91.12.5695).

If the annealing step of the PCR reaction is too low, the primers interact with the template in an unspecific manner and partial match between the primer and the template will be stable enough to get unspecific amplification. Therefore, being able to influence and/or modulate the Tm temperature in a PCR reaction can be critical for successful amplification and signal generation.
The inventors found that sucrose, sorbitol, methylglucosides, trehalose, erythritol, glycine, alanine, serine, proline, xylitol and/or maltose influenced the Tm.

The invention also relates to a method of amplifying a nucleic acid sample comprising the steps of,
a. providing a lyophilized composition comprising,
   i. a first reagent mixture for performing a nucleic acid amplification reaction,
   ii. and an amplification adjuvant selected from the group of sucrose, sorbitol, methylglucosides, glycine, arginine, proline, erythritol, xylitol, maltose, lactose, Ficoll, mannitol, dextran, trehalose, alanine and serine, or any combination thereof,
b. re-hydrating the lyophilization cakes,
c. optionally adding one or more oligonucleotide primers depending on whether these are present in the first reagent mixture,
d. adding a sample, and
e. performing an amplification reaction.

The invention also relates to a method of amplifying a nucleic acid sample comprising the steps of,
a. providing a lyophilized composition comprising,
   i. a first reagent mixture for performing a nucleic acid amplification reaction,
   ii. and an amplification adjuvant selected from the group of sucrose, sorbitol, methylglucosides, glycine, arginine, proline, erythritol, xylitol, maltose, lactose, Ficoll, mannitol, dextran, and trehalose.
b. re-hydrating the lyophilization cakes,
c. optionally adding one or more oligonucleotide primers depending on whether these are present in the first reagent mixture,
d. adding a sample, and
e. performing an amplification reaction.

Standard amplification concentrations are used.

Water is added as described below. As an example, only we quote as follows:
A 10×PCR Buffer is ideally used. It will comprise necessary salts.
A deoxynucleotide mix is used usually these are around 200 μM.
Primers are added.
Taq DNA Polymerase is used, e.g. at 0.05 units/μl
25 mM MgCl₂ is used.
The primers may have a concentration of 0.1 to 0.5 μM. While the template DNA is usually around 200 pg/μl in the present invention the amount of target DNA might be very low.

Preferably, the amplification is done in a total volume of over 19 μl, over 22 μl, over 25 μl, between 5 μl and 30 μl, or between 5 μl and 100 μl. This allows the excipient to exhibit maximum effect.

The invention also relates in a preferred embodiment to a method as outlined above wherein the rehydration of the lyophilization cakes and the addition of the sample are done in one step and the rehydration fluid and sample volume together is in the range of about 5 to 100 microliters of volume. Preferably the total volume is in the range of 15 to 80 microliters, more preferably in the range of 15 to 60 microliters more preferably in the range of 15 to 40 microliters and most preferably in the range of 5 to 30 microliters. As outlined above the inventors have astonishingly found that these larger volumes of amplification mixture make it possible to amplify in particular difficult forensic samples that might comprise trace amounts of target nucleic acid.

The invention thus relates to a method of amplifying a nucleic acid sample comprising the steps of,
a. providing a lyophilized composition comprising,
   i. a first reagent mixture for performing a nucleic acid amplification reaction including primers,
   ii. and an amplification adjuvant selected from the group of sucrose, sorbitol, methylglucosides, glycine, arginine, proline, erythritol, xylitol, maltose, lactose, Ficoll, mannitol, dextran, trehalose, alanine and serine, or any combination thereof,
b. re-hydrating the lyophilization cakes with a mixture of water and sample, and
c. performing an amplification reaction.

The invention thus relates to a method of amplifying a nucleic acid sample comprising the steps of,
a. providing a lyophilized composition comprising,
   i. a first reagent mixture for performing a nucleic acid amplification reaction including primers,
   ii. and an amplification adjuvant selected from the group of sucrose, sorbitol, methylglucosides, glycine, arginine, proline, erythritol, xylitol, maltose, lactose, Ficoll, mannitol, dextran and trehalose,
b. re-hydrating the lyophilization cakes with a mixture of water and sample, and
c. performing an amplification reaction.

Herein step b) has 10 to 100 microliters of volume, preferably the total volume is in the range of 15 to 80 microliters, more preferably in the range of 15 to 60 microliters more preferably in the range of 10 to 40 microliters and most preferably in the range of 5 to 30 microliters.

Thus, in a particularly preferred embodiment the forensic sample is added to the rehydration fluid, e.g., water and the volume thereof is, as outlined above, between 10 to 100 microliters. Exemplary samples have been designated and outlined herein. In the forensic field such difficult samples might be blood or sperm from crime sites containing only trace amounts of target nucleic acid.

The invention relates to a method of amplifying a nucleic acid forensic sample comprising the steps of,
a. providing a lyophilized composition comprising,
   i. a first reagent mixture for performing a nucleic acid amplification reaction including primers,
   ii. and an amplification adjuvant selected from the group of sucrose, sorbitol, methylglucosides, glycine, arginine, proline, erythritol, xylitol, maltose, lactose, Ficoll, mannitol, dextran, trehalose, alanine and serine, or any combination thereof,
b. re-hydrating the lyophilization cakes with a mixture of water and sample, and
c. performing an amplification reaction, wherein the sample comprises a mixture of background nucleic acids and target nucleic acids and the ratio is

| target DNA (pg/μl) | background DNA (pg/μl) |
|---|---|
| 1 | 100 |
| 1 | 1000 |
| 1 | 10000 |
| 1 | 100000 |
| 1 | 1000000 |

The invention relates to a method of amplifying a nucleic acid forensic sample comprising the steps of,
a. providing a lyophilized composition comprising,
   i. a first reagent mixture for performing a nucleic acid amplification reaction including primers,
   ii. and an amplification adjuvant selected from the group of sucrose, sorbitol, methylglucosides, glycine, arginine, proline, erythritol, xylitol, maltose, lactose, Ficoll, mannitol, dextran and trehalose,
b. re-hydrating the lyophilization cakes with a mixture of water and sample, and
c. performing an amplification reaction, wherein the sample comprises a mixture of background nucleic acids and target nucleic acids and the ratio is

| target DNA (pg/μl) | background DNA (pg/μl) |
|---|---|
| 1 | 100 |
| 1 | 1000 |
| 1 | 10000 |
| 1 | 100000 |
| 1 | 1000000 |

In such a method for example male DNA is amplified in the presence of a large amount of female DNA background, or vice versa. Hence, the method preferably relates to such mixtures of male and female DNA samples. Here, sample amount of true target is scarce, and the present method has solved this problem.

For different excipients different concentrations are preferred. The inventors have evaluated the preferred concentration range for 20 microliters of final PCR reagent mixture. These values are given below.

For all of the excipients listed below the lower range of concentrations in 20 microliters is typically 2%.

| List excipients | Preferred input | Max. input conc. in % |
|---|---|---|
| sorbitol | 14 to 60% | 59.3 |
| methylglucosides | 10 to 40% | 42.3 |
| glycine | 2 to 17% | 16.9 |
| arginine | 2 to 13% | 12.7 |
| proline | 4 to 28% | 27.9 |
| trehalose | 6 to 43% | 42.3 |
| sucrose | 12 to 54% | 53.3 |
| dextran | 1 to 26% | 25.4 |
| erythritol | 4 to 43% | 42.3 |
| xylitol | 4 to 54% | 53.3 |
| maltose | 4 to 43% | 42.3 |
| lactose | 2 to 17% | 16.9 |
| Ficoll | 2 to 17% | 16.9 |
| mannitol | 2 to 17% | 16.9 |

Additionally, the inventors have further evaluated the preferred concentration range for 20 microliters of final PCR reagent mixture for alanine and serine. These values are given below. Again, the lower range of concentrations in 20 microliters is typically 2% w/v.

| List excipients | Preferred input (w/v) | Max. input conc. in % w/v |
|---|---|---|
| alanine | 2 to 14% | 13.6 |
| serine | 2 to 30% | 29.6 |

The invention thus relates also to a composition, wherein the concentration of the adjuvant/excipient in the lyophilized compositions is as follows,

| excipient | % |
|---|---|
| sorbitol | 14 to 60% |
| methylglucosides | 10 to 40% |
| glycine | 2 to 15% |
| arginine | 2 to 15% |
| proline | 4 to 18% |
| trehalose | 6 to 18% |
| sucrose | 12 to 22% |
| dextran | 1 to 4% |
| erythritol | 6 to 20% |
| xylitol | 6 to 22% |
| maltose | 6 to 22% |
| lactose | 2 to 10% |
| Ficoll | 2 to 10% |
| mannitol | 2 to 10% |

and preferably, no glycerol is in the mixture.

The invention thus relates also to a composition, wherein the concentration of the adjuvant/excipient in the lyophilized compositions is as follows,

| excipient | % |
|---|---|
| sorbitol | 14 to 60% |
| methylglucosides | 10 to 40% |
| Glycine | 2 to 15% |
| Arginine | 2 to 15% |
| Proline | 4 to 18% |
| trehalose | 6 to 18% |
| Sucrose | 12 to 22% |
| Dextran | 1 to 4% |
| erythritol | 6 to 20% |
| Xylitol | 6 to 22% |
| Maltose | 6 to 22% |
| Lactose | 2 to 10% |
| Ficoll | 2 to 10% |
| mannitol | 2 to 10% |

and preferably, no glycerol is in the mixture.

The invention relates also to a composition, wherein the concentration of the adjuvant/excipient in the lyophilized compositions is as follows,

| excipient | % (w/V) |
|---|---|
| sorbitol | 14 to 60% |
| methylglucosides | 10 to 40% |
| glycine | 2 to 15% |
| arginine | 2 to 15% |
| proline | 4 to 18% |
| trehalose | 6 to 18% |
| sucrose | 12 to 22% |
| dextran | 1 to 4% |
| erythritol | 6 to 20% |
| xylitol | 6 to 22% |
| maltose | 6 to 22% |
| lactose | 2 to 10% |
| Ficoll | 2 to 10% |
| mannitol | 2 to 10% |
| alanine | 2 to 14% |
| serine | 2 to 15% |

and preferably, no glycerol is in the mixture.

The invention thus relates more preferably also to a composition, wherein the concentration of the adjuvant/excipient in the lyophilized compositions is as follows,

| excipient | % |
|---|---|
| sorbitol | 17 to 19% |
| methylglucosides | 14 to 16% |
| sucrose | 17 to 19% |
| dextran | 1 to 3% |
| erythritol | 13 to 16% |
| xylitol | 17 to 19% |
| maltose | 14 to 16%. |

The invention relates also to a composition, wherein the concentration of the adjuvant/excipient in the lyophilized compositions is as follows,

| excipient | % (w/v) |
|---|---|
| sorbitol | 17 to 19% |
| methylglucosides | 14 to 16% |
| sucrose | 17 to 19% |
| dextran | 1 to 3% |
| erythritol | 13 to 16% |
| xylitol | 17 to 19% |
| maltose | 14 to 16% |
| trehalose | 6 to 18% |
| glycine | 3 to 4% |
| mannitol | 5 to 8% |
| alanine | 3 to 10% |
| serine | 3 to 10% |

The invention thus relates also to a composition, wherein the concentration of the adjuvant/excipient in the lyophilized compositions is as follows,

| excipient | % (w/v) |
|---|---|
| sorbitol | 18% |
| methylglucosides | 15% |
| Glycine | 3%, or 6% |
| Proline | 4%, or 4.95%, or 9.9% |
| trehalose | 6%, or 7.5%, or 13.5%, or 15%, or 20% |
| Sucrose | 13.5%, or 16%, or 18%, or 18.9% |
| Dextran | 1%, or 2.25% |
| erythritol | 7.5%, or 15% |
| Xylitol | 9.45%, or 18%, or 18.9% |
| Maltose | 7.5%, or 15% |
| Lactose | 3%, or 6% |
| Ficoll | 3%, or 6% |
| mannitol | 3%, or 6%, or 8% |
| alanine | 4% |
| serine | 4% |

and preferably, no glycerol is in the mixture.

The assay makes use of locus-specific primers. Preferred primers have a length of about 15-100, more preferably about 20-50, most preferably about 20-40 bases. It is essential that the primers of the method span the region comprising the target sequence. The target nucleic acid sequence is amplified by using the nucleic acid containing that sequence as a template. If the nucleic acid contains two strands, it is necessary to separate the strands of the nucleic acid before it can be used as the template, either as a separate step or simultaneously with the synthesis of the primer extension products. This strand separation can be accomplished by any suitable denaturing method including physical, chemical, or enzymatic means. One physical method of separating the strands of the nucleic acid involves heating the nucleic acid until it is completely (>99%) denatured. Typical heat denaturation may involve temperatures ranging from about 80° C. to 105° C., preferably about 90° C. to about 98° C., still more preferably 93° C. to 95° C., for times ranging from about 1 to 10 minutes. In the case of isothermal amplification, the strand separation may also be induced by an enzyme from the class of enzymes known as helicases or the enzyme RecA, which has helicase activity and is known to denature DNA.

This amplification reaction can be performed using any suitable method. Generally, it occurs in a buffered aqueous solution. In some preferred embodiments, the buffer pH is about 7.5-8.9 at 25° C. Preferably, a molar excess of the oligonucleotide primers is added to the buffer containing the separated template strands. It is understood, however, that the amount of complementary strand may not be known if the process herein is used for some applications, so that the amount of primer relative to the amount of complementary strand cannot be determined with certainty. As a practical matter, however, the amount of primer added, will generally be in molar excess over the amount of complementary strand (template) when the sequence to be amplified is contained in a mixture of complicated long-chain nucleic acid strands. A large molar excess is preferred to improve the efficiency of the process.

Of course, further primer pairs can be present in the reaction for amplifying also other regions of interest.

The means and methods of the present invention comprise the use of nucleic acid probes. A nucleic acid probe according to the present invention is an oligonucleotide, nucleic acid or a fragment thereof, which is substantially complementary to a specific nucleic acid sequence.

Suitable hybridization probes include the LightCycler probe (Roche), the TaqMan probe (Life Technologies), a molecular beacon probe, a Scorpion primer, a Sunrise primer, a LUX primer and an Amplifluor primer. TaqMan probes are preferred in the context of the present invention.

Any source of nucleic acid, in purified or non-purified form, can be utilized as the starting nucleic acid, if it contains or is thought to contain the target nucleic acid sequence desired. It may also be RNA. Thus, the process may employ, for example, DNA which may be single stranded or double stranded. In addition, a DNA-RNA hybrid which contains one strand of each may be utilized. A mixture of any of these nucleic acids may also be employed, or the nucleic acids produced from a previous amplification reaction using the same or different primers may be utilized. The nucleic acid amplified is preferably DNA. The target nucleic acid sequence to be amplified may be only a fraction of a larger molecule or can be present initially as a discrete molecule, so that the target sequence constitutes the entire nucleic acid. It is not necessary that the target sequence to be amplified be present initially in a pure form; it may be a minor fraction of a complex mixture or a portion of nucleic acid sequence, since a particular organism of interest might constitute only a very minor fraction of a particular biological sample. The starting nucleic acid may contain more than one desired target nucleic acid sequence which may be the same or different. The nucleic acid(s) may be obtained from any source and include plasmids and cloned DNA, DNA from any source, including viruses, bacteria, archaea, and eukaryotic organisms, e.g., yeast, molds, algae, plants or animals. DNA may be extracted from, for example, blood or other fluids, or tissue material such as chorionic villi or amniotic cells by a variety of techniques such as that described by Maniatis et al., Molecular Cloning: A Laboratory Manual, (New York: Cold Spring Harbor Laboratory) pp 280-281 (1982).

A sample is added prior to the amplification. The term “sample” as used herein refers to a sample of bodily fluid obtained for the purpose of diagnosis, prognosis, or evaluation of a subject of interest, such as a patient. Preferred test samples include blood, serum, plasma, cerebrospinal fluid, urine, saliva, skin, hair cells, sputum, bone, sperm, vaginal swaps, and pleural effusions. In addition, one of skill in the art would realize that some test samples would be more readily analyzed following a fractionation or purification procedure, for example, separation of whole blood into serum or plasma components.

Thus, in a preferred embodiment of the invention the sample is selected from the group comprising a blood sample, a serum sample, a plasma sample, a cerebrospinal fluid sample, a saliva sample and a urine sample or an extract of any of the aforementioned samples. Preferably, the sample is serum or a plasma or a crime scene sample, most preferably a crime scene sample. It may the sample used directly or the nucleic acid isolated from the sample. Ideally, the nucleic acid is isolated. Preferably the sample is a sperm sample. Preferably it is DNA as a sample, but it may be RNA.

Preferably, in the method according to the invention the method is a non-isothermal amplification reaction or an isothermal amplification reaction. Most preferably herein it is non-isothermal.

Preferably, in the method according to the invention the method is a polymerase chain reaction amplification reaction (PCR) and preferably a real-time PCR.

A real-time polymerase chain reaction (real-time PCR, or qPCR) is a laboratory technique of molecular biology based on the polymerase chain reaction (PCR). It monitors the amplification of a targeted DNA molecule during the PCR (i.e., in real time), not at its end, as in conventional PCR. Real-time PCR can be used quantitatively (quantitative real-time PCR) and semi-quantitatively (i.e., above/below a certain amount of DNA molecules) (semi-quantitative real-time PCR). Two common methods for the detection of PCR products in real-time PCR are (1) non-specific fluorescent dyes that intercalate with any double-stranded DNA and (2) sequence-specific DNA probes consisting of oligonucleotides that are labelled with a fluorescent reporter, which permits detection only after hybridization of the probe with its complementary sequence. “RT-PCR” commonly denotes reverse transcription polymerase chain reaction and not real-time PCR. Diagnostic qualitative PCR is applied to rapidly detect nucleic acids that are diagnostic of, for example, infectious diseases, cancer and genetic abnormalities. The introduction of qualitative PCR assays to the clinical microbiology laboratory has significantly improved the diagnosis of infectious diseases, and is deployed as a tool to detect newly emerging diseases, also as new strains of flu and coronavirus, in diagnostic tests. Also forensics are an application. The present invention may be used with these techniques. Hence, we claim a method using also these techniques.

The probes are designed to bind to a specific region on one strand of a template nucleic acid. Following annealing of the PCR primer to this strand, Taq enzyme extends the DNA with 5′ to 3′ polymerase activity. Taq enzyme also exhibits 5′ to 3′ exonuclease activity. TaqMan™ probes consist of a fluorophore covalently attached to the 5′-end of the oligonucleotide and a quencher at the 3′-end-to repress a fluorescence emission after excitation- and are protected at the 3′ end by phosphorylation to prevent them from priming Taq extension. If the TaqMan™ probe is hybridised to the product strand, an extending Taq molecule will hydrolyse the probe and separating the quencher from the fluorophore. This means that there is no quenching of the fluorophore signal any longer and a fluorescent signal is emitted. This signal can be used as the basis of detection. The signal in this instance is cumulative. Taqman probes and their mode of function are known to the person skilled in the art.

Hybridization probes are available in a number of forms and these may also be included in the compositions. Molecular beacons are oligonucleotides that have complementary 5′ and 3′ sequences such that they form hairpin loops. Terminal fluorescent labels are in close proximity for FRET to occur when the hairpin structure is formed. Following hybridisation of molecular beacons to a complementary sequence the fluorescent labels are separated, so FRET does not occur, and this forms the basis of detection during a polymerase chain reaction.

In such a real-time method probes and primers are labelled with a labelling system. In the context of the present invention, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, auch as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like. Fluorescent dyes are preferred.

An optional additional component of the composition of the invention is an antioxidant and/or anti-maillard agent. A particular example of such a reagent is threonine such as L-threonine although others may be used. These eliminate any oxygen produced and therefore assist in the stabilization of the reaction mixture. They are suitably added in an amount which does not affect the pH of the composition, as determined by the buffer, which is generally between pH is about 7.5-8.9 at 25° C. (but pH is dependent on buffers). The amount of antioxidant which can therefore be added will depend upon the nature of the antioxidant itself. For example, for threonine it may be present in the composition in an amount of from 2-10 mM, for example at about 2.5 mM

Compositions of the invention may further comprise an RNase inhibitor. The applicants have surprisingly found that addition of RNase inhibitors has a stabilizing effect on the composition, even where the composition contains no RNA elements or is intended for use in amplification reactions in which RNA is involved, such as RT-PCR. Its addition improves the stability of the composition, even over prolonged time periods, at the end of which, the composition is still able to operate in an effective manner when used in real-time PCR methods.

Thermophiles make great polymerases which are preferred herein. They may be aerobic or anaerobic, and are found in a wide variety of genera and species, including the phototrophic bacteria (e.g., the purple bacteria, green bacteria, and cyanobacteria), eubacteria (e.g., Bacillus, Clostridium, Thiobacillus, Desulfotomaculum, Thermus, lactic acid bacteria, actinomycetes, spirochetes, and numerous other genera), and the archaebacteria (e.g., Pyrococcus, Thermococcus, Thermoplasma, Thermotoga, Sulfolobus, and the methanogens). Accordingly, the environments in which thermophiles are normally found vary greatly, although all of these areas are associated with high temperatures.

Thermophiles, like other bacteria, contain five types of DNA polymerases, termed polymerase I, II, III, IV, and V. Given the nature of thermophile habitats, these enzymes typically exhibit thermostability, and are generally referred to as thermostable DNA polymerases

Thermostable DNA polymerases have proven very useful in a number of applications in molecular biology. One such application is the polymerase chain reaction (PCR). A number of applications, for example long range PCR, are hindered by the error rates of Pol I proteins currently available (e.g., Taq DNA Pol I). In addition to decreased error rates, a number of applications would benefit from the use of DNA Pol I exhibiting improved sequence discrimination activity, primer mismatch tolerance, and increased thermostability. For example, a DNA Pol I that tolerates primer mismatches would be useful in PCR methods involving the use of degenerative primers. Polymerases commonly used for PCR are obtained from various thermophilic micro-organisms: Thermus aquaticus (Taq), Pyrococcus furiosus (Pfu polymerase), Thermococcus litoralis (Wind or Tli polymerase or Vent polymerase) and Thermus thermophilus (Tth polymerase).

In the reaction according to the invention preferably the amplicons are located on one or more template nucleic acids of the sample and the amount of the one or more template nucleic acids in said nucleic acid amplification reaction mixture is between 1 fg to 5 μg, more preferably between 100 fg to 1 μg, even more preferably between 500 fg to 500 ng, even more preferably between 750 fg to 250 ng, even more preferably between 850 fg to 250 ng, even more preferably between 900 fg to 150 ng, even more preferably between 1 μg to 150 ng and most preferably between 2 μg to 110 ng. DNA/RNA concentrations claimed herein are more preferably, from 0.001 pg/μl to 1 ng/μl. Preferred is a range of 0.5 pg/μl to 0.02 pg/μl. The inventive method preferably claims 0.3 pg/μl to 0.03 pg/μl of DNA/RNA where normal mixes fail.

Herein, a “nucleic acid” may be inter alia, RNA, DNA, cDNA (complementary DNA), ccfDNA (circulating cell-free), LNA (locked nucleic acid), mRNA (messenger RNA), mtRNA (mitochondrial), rRNA (ribosomal RNA), tRNA (transfer RNA), nRNA (nuclear RNA), siRNA (short interfering RNA), snRNA (small nuclear RNA), snoRNA (small nucleolar RNA), scaRNA (Small Cajal Body specific RNA), microRNA, dsRNA (double-stranded RNA), ribozyme, riboswitch, viral RNA, dsDNA (double-stranded DNA), ssDNA (single-stranded DNA), plasmid DNA, cosmid DNA, chromosomal DNA, viral DNA, mtDNA (mitochondrial DNA), nDNA (nuclear DNA), snDNA (small nuclear DNA) or the like or any other class or sub-class of nucleic acid which is distinguishable from the bulk nucleic acid in a sample.

More importantly, the method according to the invention works for difficult forensic samples comprising mixtures of DNA samples from multiple donors, such as samples containing male and female DNA.

Ideally and preferably, the one or more template nucleic acids are extracted from a forensic sample containing biological material, sputum, saliva, blood, hairs, hair follicles, sperm, vaginal secretions, liquor, blood plasma, blood serum, fingernails, tissue, urine, plants, microbes, bacteria, viruses, any other parts of the human or animal body or from any other sample containing biological material from which nucleic acids can be extracted.

The samples are preferably forensic samples. Preferably, they are contaminated with other DNA from third party individuals or inhibitors.

Preferably, amplification is done in a total volume of over 5 μl, preferably over 19 μl, over 22 μl, over 25 μl, between 5 μl and 30 μl, or between 5 μl and 100 μl.

Preferably, amplification of forensic samples is done in a total volume of over 5 μl, preferably over 19 μl, over 22 μl, over 25 μl, between 5 μl and 30 μl, or between 5 μl and 100 μl.

As outlined above the invention relates to the interplay of amplification volume and beneficial excipient effect more importantly for forensic samples and amplifications.

More preferably the amplification reaction is a PCR. Most preferably, the amplification reaction is a multiplex PCR, i.e. the amplification of more than one target nucleic acid sequence in a single tube, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 loci (target nucleic acid sequences) may be amplified simultaneously.

Preferably, the method works with mixtures of the following ratios:

| First DNA (pg/μl) | Second DNA (pg/μl) |
|---|---|
| 1 | 100 |
| 1 | 1000 |
| 1 | 10000 |
| 1 | 100000 |
| 1 | 1000000 |

The example herein shows, 1:400000; 1 pg/μl male DNA+400 ng/μl female DNA as working well with the present invention. Here 1 μg was detected in a background of 400 ng.

Claims herein are therefore difficult amplifications where the ratio might be 1:10000 or 1:100000.

In a preferred embodiment the target in the sample is a human multi copy gene.

In existing liquid-based master mixes sample volume to be added is limited in volume. So in forensic based applications it might well be that rare targets are not amplified as the addition of 20 μl of sample would be needed to ensure the addition of a target molecule.

We disclose an example of 10 μl final amplification volume.

For a 5× liquid PCR master mix this means 2 μl master mix are added and 8 μl sample may be added. For a 10× liquid PCR master mix this means 1 μl volume of mix is added and 9 μl sample may be added. This is not much. Our invention allows the addition of higher sample volumes which solves a big problem.

The present invention therefore relates to a method wherein, the added sample makes up about more than 50% of the total amplification volume, more than 60% of the total amplification volume, more than 70% of the total amplification volume, more than 80% of the total amplification volume, more than 90% of the total amplification volume, more than 92% of the total amplification volume, or more than 95% of the total amplification volume. The added sample can also make up about 100% of the total amplification volume. The aforementioned percentages for the sample are expressed as % v/v of the total amplification volume.

The invention also relates to a method of preparing a lyophilized nucleic acid amplification composition comprising the steps, mixing in liquid form of
a. a first reagent mixture for performing a nucleic acid amplification or elongation reaction,
b. an amplification/elongation adjuvant preferably selected from the group of sucrose, sorbitol, methylglucosides, glycine, arginine, proline, erythritol, xylitol, maltose, lactose, Ficoll, mannitol, dextran, trehalose, alanine and serine, or any combination thereof, and
lyophilizing the composition.

The invention also relates to a method of preparing a lyophilized nucleic acid amplification composition comprising the steps, mixing in liquid form of
a. a first reagent mixture for performing a nucleic acid amplification or elongation reaction,
b. an amplification/elongation adjuvant preferably selected from the group of sucrose, sorbitol, methylglucosides, glycine, arginine, proline, erythritol, xylitol, maltose, lactose, Ficoll, mannitol, dextran and trehalose, and
lyophilizing the composition.

The freeze-drying protocol used will depend to some extent upon the particular composition being dried and will be determined in each case using routine procedures. Typically, the composition will be subject to a freezing step in which it is cooled to a low temperature for example from about −20° C. to −60° C. and generally at about −40° C. at a pressure of from 300-400 torr (the person skilled in the art is readily able to calculate mBar), and held at this temperature for a sufficient period of time to ensure that complete freezing occurs. The pressure is then reduced to an appropriate level depending upon the particular freeze-dryer used. Some may operate a pressures as low as 6 mTorr but for current purposes, pressures of from 10 to 100 mTorr may be suitable to allow the water to sublimate. Suitably then the composition is brought gradually back up to room temperature under reduced pressure, before the vacuum is released to minimize condensation effects. Optionally, the vacuum is released in the presence of an inert atmosphere such as under nitrogen, so that the product is maintained in an inert environment. This also prevents moisture ingress. Freeze-dried product obtained in this way is suitably packaged immediately for example in foil wrappers, to minimize contamination risk. If the composition is contained within containers such as reagent pots, these are suitably sealed before vacuum is released.

Care needs to be taken to ensure that all reagents utilized in the composition do not contain materials or contaminants which could inhibit or prevent freeze drying in the levels in which they are found. Thus, for example, it may be necessary to remove substances such as glycerol which are sometimes included in commercially available enzymes such as polymerases, reverse transcriptase polymerases and RNase inhibitors, and or to reduce the levels of substances such as dimethyl sulfoxide (DMSO) which may be found in intercalating dyes which may be used as DNA duplex binding agents.

In still further aspects, a lyophilized pellet of the embodiments further comprises a stabilizer (e.g., protein stabilizer such as bovine serum albumin (BSA) or gelatine). In certain specific aspects, a lyophilized pellet of the embodiments is formed from an aqueous mixture comprising about 0.1 to about 1.0 mg/ml of a polypeptide stabilizer such as BSA. For example, the pellet can be formed from an aqueous mixture comprising about 0.5 mg/ml of BSA. Herein, “pellet” and “cake” are used interchangeably.

In still another aspect the invention relates to freeze drying the amplification mixture. In this method a number of steps are performed 1) adjustment of the sample temperature 2) slow controlled freezing of samples 3) maintaining the freezing temperature 4) setting primary drying conditions 5) holding of primary drying conditions 6) slow controlled setting of temperature for secondary drying conditions 7) setting of vacuum for secondary drying conditions 8) final drying. Freezing is done for about four to seven hours. The temperature selected is from about minus 20° C. to about minus 80° C. Drying is done for about 8 to 16 hours. A second drying step is required or preferred and will be performed for about 20 to 40 minutes. Vacuum pressure in mbar is between 0.5 and 0.004. Initial drying is done at cold temperatures.

| | StaWrt | Freeze | | |
|---|---|---|---|---|
| | 1 | 2 | 3 | 4 |
| Temp, | 10 | 10 | | (—)30/−52/−60 |
| ° C. | | | | |
| Vacuum, | off | off | off | off |
| mbar | | | | |
| Time, | --:-- | 00:00:00/ | 00:30:00/ | 00:30:00/ |
| h. min | | 00:15:00/ | 02:30:00/ | 04:00:00/ |
| | | 00:30:00 | 04:00:00 | 06:00:00 |
| Alarm, | off | off | off | off |
| mbar | | | | |
| | 00:00 | 00:15 | 02:45 | 06:45 |

1st drying

| 5 | 6 | 7 | 8 |
|---|---|---|---|
| (—)30/−52/−60 | (—)30/−52/−60 | 20 | 20 |
| 0.63/0.14/ | 0.63/0.14/ | 0.63/0.14/ | 0.63/0.014/ |
| 0.0047 | 0.0047 | 0.0047 | 0.0047 |
| 00:00:00/ | 02:00:00/ | 00:15:00/ | 00:00:10/ |
| 00:01:00/ | 16:00:00/ | 02:00:00/ | 00:01:00/ |
| 00:015:00 | 20:00:00 | 04:00:00 | 00:05:00 |
| off | off | 1.650 | 1.650 |
| 06:46 | 22:46 | 24:46:00 | 24:47:00 |

2nd drying
9
20
0.63/0.014/0.0047
00:015:00/05:00:00/08:00:00
1.650
29:47:00
1 Start
2 Adjustment of the sample temperature
3 Slow controlled freezing of samples
4 Maintaining the freezing temperature
5 Setting primary drying conditions
6 Hold of primary drying conditions
7 Slow controlled setting of temperature for secondary drying conditions
8 Setting of vacuum for secondary drying conditions
9 Sec.(final) drying

Preferred volumes are 8 μl to 12 μl preferably 10 μl of a 2× master mix for use in lyophilization.

### Volumes for PCR

The cake is later hydrated and the preferred PCR mix volumes are from 2 μl to 100 μl, preferably, 10 μl to 80 μl, more preferably 5 μl to 30 μl.

### High Resistance to High Air Humidity and Stability of Lyophilized Cake Structures.

Humidity can be a limiting factor for lyophilized PCR mixes, since the lyophilized cakes can start to rehydrate when exposed to high relative humidity. Especially for PCR mixes which have been lyophilized in plate format, e.g. a 96 well PCR plate, high relative humidity can be a challenging and limiting factor. Once the lyophilized product is opened and exposed to high humidity environment the time for pipetting either manually or on automated platforms could be limiting since the cakes could start to collapse and rehydrate and by that become difficult or even impossible to redissolve and/or the redissolved cakes attach the pipette tips which may lead to cross contamination or failure of the experiment, especially in automated platforms. This could render such a product useless.

Surprisingly the inventors have found that some excipients produce cakes with high stability at relative humidity (higher than 50%). In particular, mannitol-based cakes with 4-16% w/v mannitol, preferably 4-10% w/v mannitol, more preferably 4-9% w/v and most preferably 5-8% w/v mannitol in the final reaction, or glycine-based cakes with 1-5% w/v glycine, more preferably 2-4% w/v glycine and most preferably 3-4% w/v glycine in final concentration reaction mix have shown high resistance and stability at relative humidity higher than 50% for at least one hour with no negative impact on cake structure and stability (FIG. 6). In a preferred embodiment, the final reaction mix further comprises 0.5-3% w/v sucrose, preferably 1-2% w/v sucrose and/or 0.5-2% w/v xylitol, preferably 1% w/v xylitol and/or 1-3% w/v trehalose, most preferably 1.5% w/v trehalose.

### FIGURE CAPTIONS

FIG. 1

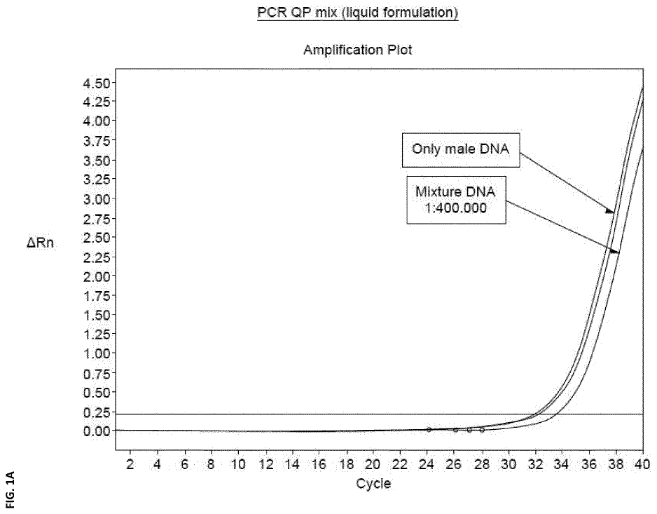

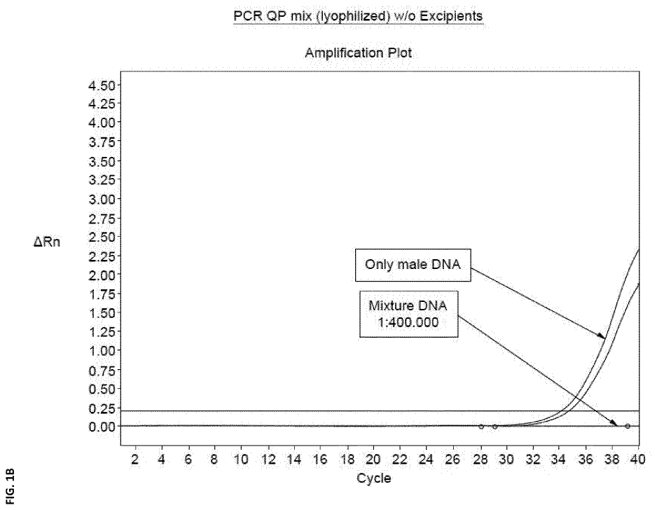

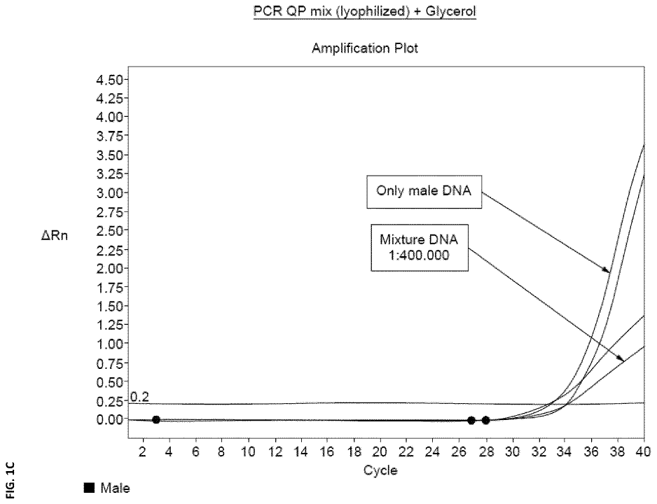

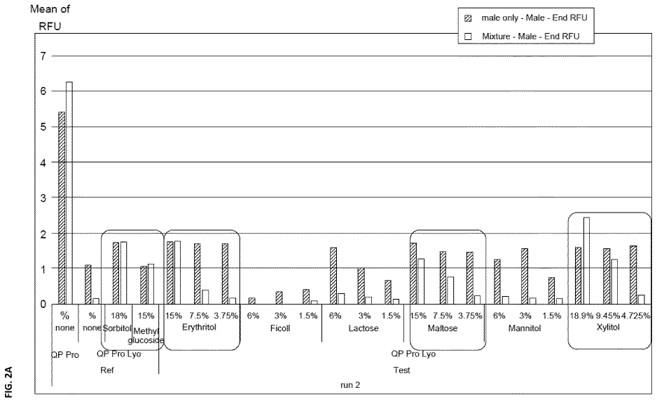

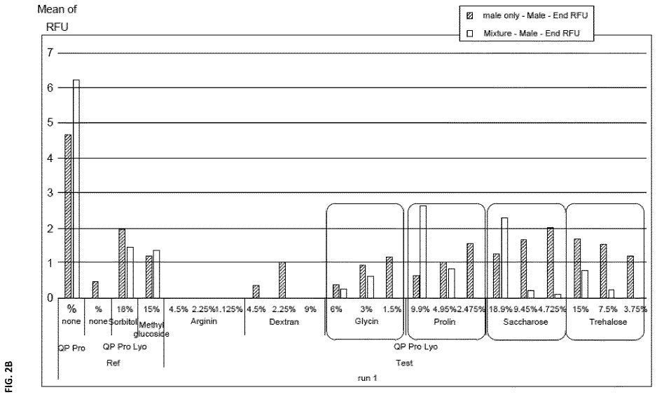

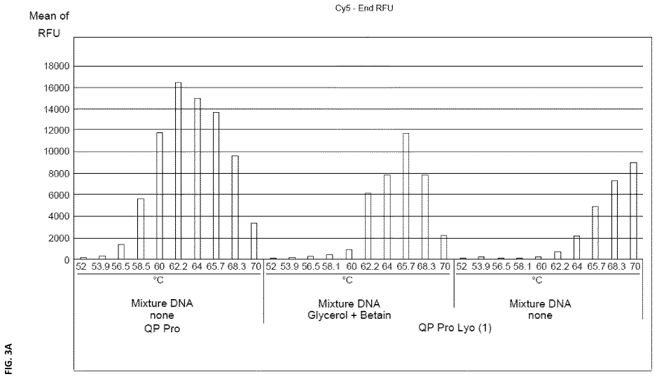

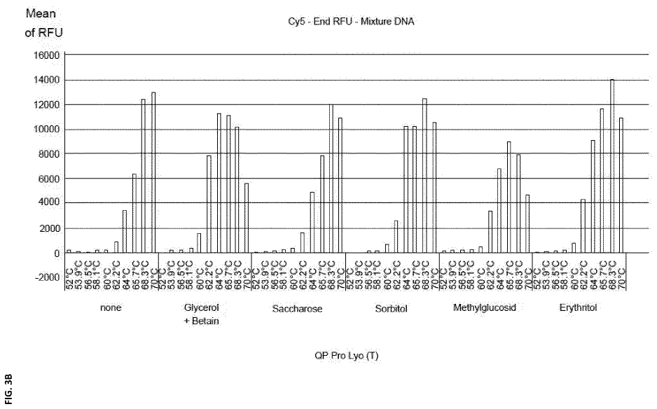

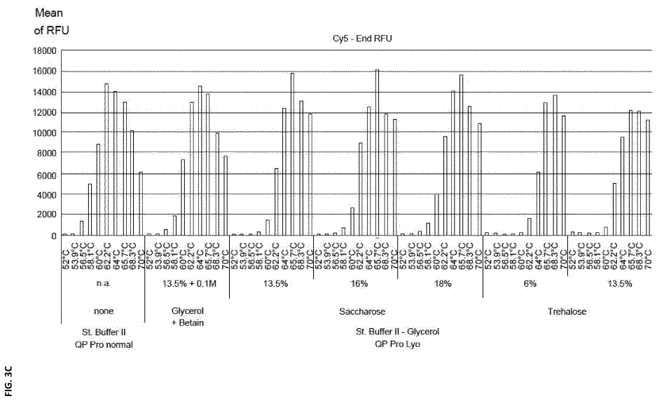

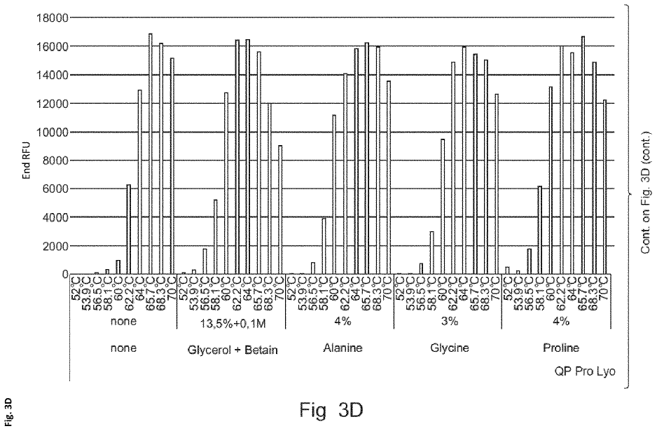

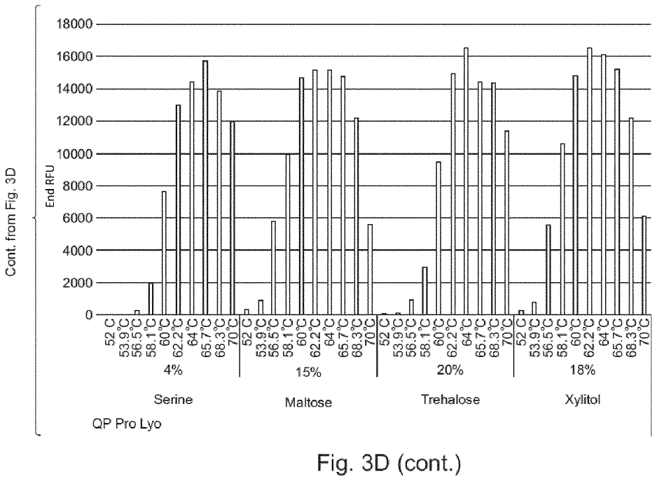

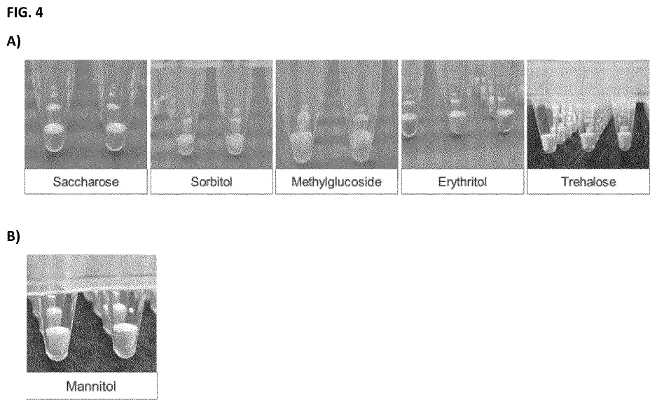

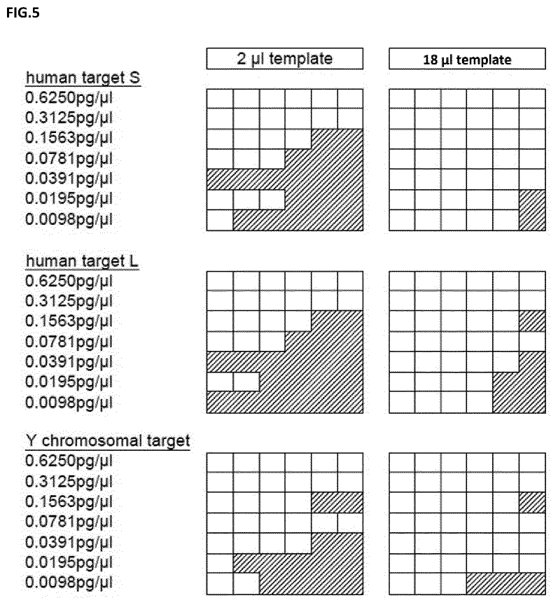

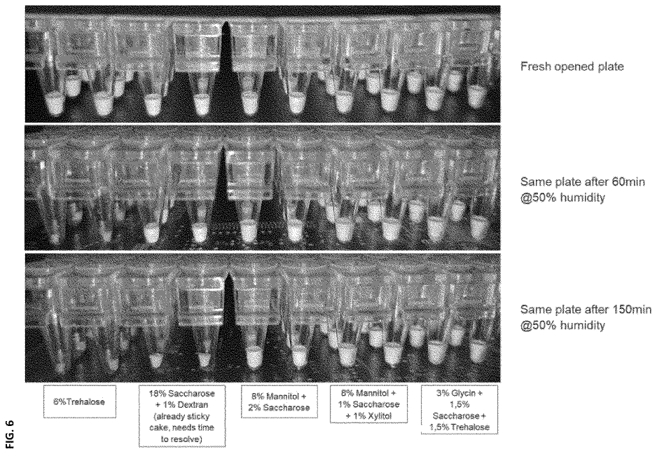

qPCR Amplification curves for Y chromosomal multicopy target. Human male DNA has been amplified either pure (1 pg/μl) or in a mixture with human female DNA (1:400000; 1 pg/μl male DNA+400 ng/μl female DNA). Arrows indicate amplification curves of Y chromosomal multicopy target either in pure samples or mixture samples. (A) A liquid-based PCR formulation containing glycerol and betaine (QP mix) has been used for amplification. Y chromosomal multicopy target has been successfully amplified and detected in both conditions (pure and in mixture). (B) A lyophilizable version of QP mix (without glycerol and betaine) has been used for amplification. While the Y chromosomal multicopy target has been amplified successfully and detected in the pure sample detection of the Y chromosomal multicopy target has failed in the mixture sample. (C) A lyophilized version of QP mix supplemented with glycerol. Amplification and detection of the Y chromosomal multicopy target has been successfully restored in mixture samples.

FIG. 2

qPCR Amplification (delta fluorescence values) for Y chromosomal multicopy target. Human male DNA has been amplified either pure (1 pg/μl) or in a mixture with human female DNA (1:400000; 1 pg/μl male DNA+400 ng/μl female DNA). Green boxes (bars surrounded by boxes) indicate excipients which restore amplification and detection of Y chromosomal DNA in mixture samples. (A) Liquid based QP Mix containing glycerol/betaine as reference. A lyophilizable QP mix without excipients does not detect Y chromosomal multicopy target in mixture samples. Surprisingly addition of Sorbitol, methylglucosides, Erythritol, Maltose, Xylitol does restore amplification and detection of Y chromosomal multicopy target in mixture samples. (B) Liquid based QP Mix containing glycerol/betaine as reference. A Lyophilizable QP mix without excipients does not detect Y chromosomal multicopy target in mixture samples. Surprisingly addition of sorbitol, methylglucosides, glycine, proline, sucrose and trehalose does restore amplification and detection of Y chromosomal multicopy target in mixture samples.

FIG. 3

(A) Liquid based QP Mix containing glycerol/betaine as reference. Amplification for the Y chromosomal multicopy target in lyophilizable reaction mix is clearly detectable at annealing temperatures of 60° C. or lower in reactions mixes containing glycerol/betaine while the reaction mix without excipients shows a clear shift of Y chromosomal multicopy target amplification and detection towards higher annealing temperatures (62° C. or higher).

(B) Amplification of Y chromosomal multicopy target shows stronger signal amplification and detection in reaction mixes containing either glycerol/betaine, sucrose, sorbitol, methylglucosides or erythritol (wherein the excipients were used in a concentration of 13.5% w/v) and a shift towards lower annealing temperatures than the amplification of Y chromosomal multicopy target in a reaction mix without any excipients.

C) Liquid based QP Mix containing glycerol/betaine and lyophilizable QP Mix supplemented with glycerol/betaine as reference. The signal amplification of Y chromosomal multicopy target in reaction mixes containing excipients e.g. Sucrose, Trehalose is concentration dependent. The higher the concentration of the excipient is the stronger is the amplification of Y chromosomal multicopy target and the shift of amplification towards lower annealing temperatures.

D) Amplification of Y chromosomal multicopy target shows stronger signal amplification and detection in reaction mixes containing either glycerol/betaine, alanine, glycine, proline, serine, maltose, trehalose or xylitol and a shift towards lower annealing temperatures than the amplification of Y chromosomal multicopy target in a reaction mix without any excipients.

FIG. 4

A) QP mix supplemented with different excipients. Sucrose (18% w/v), sorbitol (18% w/v), methylglycoside (15% w/v), erythritol (15% w/v) and trehalose (6% w/v) support formation of lyophilization cakes which are dissolvable.

B) QP mix supplemented with mannitol as an excipient. Mannitol (8% w/v) supports formation of lyophilization cakes which are dissolvable.

Sucrose, sorbitol, methylglycosid, erythritol, trehalose, mannitol and glycine (not shown in FIG. 4) support formation of lyophilization cakes which are dissolvable.

### RATING OF LYOPHILIZATION CAKES

| Excipient | Rating (cake formation/dissolvability) |
|---|---|
| sucrose | good |
| sorbitol | good |
| methylglucosides | good |
| erythritol | good |
| trehalose | good |

A particularly nice cake is made by adding sucrose. It is easily re-hydrated. “Nice cake” in this context can be used interchangeably with “good cake”. The rating of the lyophilization cakes when using mannitol or glycine is also good.

A cake that was classified/rated as good means that it had a typical compact “cake” type 3-dimensional structure and was easily dissolvable.

### FIG. 5

qPCR amplification of three different multicopy targets in a multiplex qPCR setup using a lyophilized version of QP mix and two template volumes (2 μl vs 18 μl). Human male DNA has been diluted to concentrations indicated in the table in Example 5. Either 2 μl template DNA+16 μl H₂O or 18 μl template DNA have been applied to a lyophilized QP mix. A combination of 8% w/v mannitol, 1% w/v sucrose, and 1% w/v xylitol was used in the experiment related to this figure. PCR reactions have been run in 6 replicates for each input volume and each DNA concentration. Detected amplification is indicated with white boxes, non-detected amplification is indicated with black boxes. Higher DNA volumes result in more detected amplification events per setup and higher sensitivity for each target to be detected. 9× higher DNA volumes equal 9× higher sensitivity for detection of each amplified target.

### FIG. 6: Relative Humidity Stability of Tested Mixtures Containing Different Excipients.

Different excipient have been tested for their stabilizing effect in lyophilized cakes. Lyophilized cakes were exposed to 50% relative humidity for one hour. While the Trehalose (6% w/v)- and Sucrose (18% w/v)-based mixtures show a clear collapse at tested relative humidity, the mainly Mannitol- and Glycine-based mixtures, which contain only small amounts of sucrose and/or trehalose (≤2 percent), are not impaired in their stability when exposed to 50% relative humidity and show no cake collapse.

### EXAMPLES

### Example 1

### Influence of Glycerol on Specificity and Sensitivity

A Y-chromosomal multicopy target has been amplified and detected in a qPCR setup. Low amounts of human male DNA (1 pg/μl-2 μl volume input per reaction) have been used.

### Example 2

### Influence of Different Excipients on Specificity and Sensitivity of Amplification

A Y-chromosomal multicopy target has been amplified in a qPCR setup using a lyophilized version of QP mix in the presence of different excipients listed below. Excipients/adjuvants used were sorbitol, methylglucosides, erythritol, Ficoll, lactose, maltose, mannitol, xylitol, arginine, dextran, glycine, proline, sucrose and trehalose.

Various excipients have been tested. A tight range of maximal inputs has been identified. Various concentrations give various preferable concentration ranges in various volumes. The following is for 20 μl

| List excipients | Max. input conc. In 20 μl in % |
|---|---|
| Sorbitol | 59.3 |
| alpha-D-Methylglucoside | 42.3 |
| Glycine | 16.9 |
| Arginine | 12.7 |
| Proline | 27.9 |
| Trehalose | 42.3 |
| Sucrose | 53.3 |
| Dextran | 25.4 |
| Erythritol | 42.3 |
| Xylitol | 53.3 |
| Maltose | 42.3 |
| Lactose | 16.9 |
| Ficoll | 16.9 |
| Mannitol | 16.9 |

### Example 3

qPCR Amplification of Y chromosomal multicopy target at different annealing temperatures and in different reaction mixes supplemented with and without excipients. Human male DNA has been amplified in a mixture with human female DNA (1:400000; 1 pg/μl male DNA+400 ng/μl female DNA).

### Example 4

### Excipients Support Lyophilization Cake Formation

Lyophilizable QP mix has been supplemented with different excipients and lyophilized. Lyophilization cakes have been assessed for cake formation and dissolvability, see rating below.

### Example 5

### High Sensitivity by Maximized Input Volumes Per Reaction

Three different human multicopy targets have been amplified in a multiplex qPCR setup using a lyophilized version of QP mix. Sensitivity has been assessed for different setups using two different input volumes per PCR reaction (2 μl input volume vs. 18 μl input volume).

Human male DNA has been diluted to following template concentrations and either 2 μl template DNA+16 μl H₂O or 18 μl template DNA have been applied to a lyophilized QP mix:

DNA concentrations
0.6250 pg/μl
0.3125 pg/μl
0.1563 pg/μl
0.0781 pg/μl
0.0391 pg/μl
0.0195 pg/μl
0.0098 pg/μl

1-11. (canceled)

12. A lyophilized nucleic acid amplification composition comprising;
a. a first reagent mixture for performing a nucleic acid amplification or an elongation reaction; and
b. one or a combination of amplification/elongation adjuvants selected from the group consisting of sucrose, sorbitol, methyl glucosides, glycine, arginine, proline, erythritol, xylitol, maltose, lactose, Ficoll, mannitol, dextran, and trehalose.

13. The composition according to claim 12, wherein the first reagent mixture comprises one or more oligonucleotide primer(s) needed for nucleic acid amplification or elongation.

14. The composition according to claim 12, wherein the first reagent mixture does not comprise oligonucleotide primer(s) needed for nucleic acid amplification or elongation.

15. The composition according to claim 12, wherein:
when sucrose is present in the composition as an adjuvant, the concentration of sucrose in the composition is from about 12% to about 22% by weight;
when sorbitol is present in the composition as an adjuvant, the concentration of sorbitol in the composition is from about 14% to about 60% by weight;
when methyl glucosides are present in the composition as an adjuvant, the concentration of the methyl glucosides in the composition is from about 10% to about 40% by weight;
when glycine is present in the composition as an adjuvant, the concentration of glycine in the composition is from about 2% to about 15% by weight;
when arginine is present in the composition as an adjuvant, the concentration of arginine in the composition is from about 2% to about 15% by weight;
when proline is present in the composition as an adjuvant, the concentration of proline in the composition is from about 4% to about 18% by weight;
when erythritol is present in the composition as an adjuvant, the concentration of erythritol in the composition is from about 6% to about 20% by weight;
when xylitol is present in the composition as an adjuvant, the concentration of xylitol in the composition is from about 6% to about 22% by weight;
when maltose is present in the composition as an adjuvant, the concentration of maltose in the composition is from about 6% to about 22% by weight;
when lactose is present in the composition as an adjuvant, the concentration of lactose in the composition is from about 2% to about 10% by weight;
when FICOLL® is present in the composition as an adjuvant, the concentration of FICOLL® in the composition is from about 2% to about 10% by weight;
when mannitol is present in the composition as an adjuvant, the concentration of mannitol in the composition is from about 2% to about 10% by weight;
when dextran is present in the composition as an adjuvant, the concentration of dextran in the composition is from about 1% to about 4% by weight; and
when trehalose is present in the composition as an adjuvant, the concentration of trehalose in the composition is from about 6% to about 18% by weight.

16. The composition according to claim 12, wherein the composition does not comprise glycerol.

17. The composition according to claim 12, wherein the composition comprises at least one adjuvant selected from the group consisting of:
a. sucrose;
b. sorbitol;
C. methyl glucosides;
d. trehalose;
e. dextran; and
f. erythritol.

18. A method of amplifying a nucleic acid sample comprising:
a. providing a lyophilized first mixture composition comprising
i. reagents for performing a nucleic acid amplification reaction, and
ii. one or more amplification adjuvants selected from the group consisting of sucrose, sorbitol, methyl glucosides, glycine, arginine, proline, erythritol, xylitol, maltose, lactose, FICOLL®, mannitol, dextran and trehalose;
b. re-hydrating the lyophilized first mixture composition;
C. adding a sample to the re-hydrated lyophilized first mixture composition; and
d. performing an amplification reaction.

19. The method according to claim 18, further comprising adding one or more oligonucleotide primers to the rehydrated lyophilized first mixture composition before adding the sample, while adding the sample or after adding the sample.

20. The method according to claim 18, wherein the lyophilized first mixture composition comprises one or more oligonucleotide primers.

21. The method according to claim 18, wherein the sample is a forensic sample, and wherein the forensic sample comprises a mixture of nucleic acids.

22. The method according to claim 18, wherein the method is a non-isothermal amplification reaction.

23. The method according to claim 18, wherein the method is an isothermal amplification reaction.

24. The method according to claim 18, wherein the method is a polymerase chain reaction (PCR) amplification reaction.

25. The method according to claim 24, wherein the method is a real-time PCR amplification reaction.

26. The method according to claim 18, wherein amplification is done in a total volume of over 5 μl.

27. The method according to claim 18, wherein amplification is done in a total volume of over 19 μl.

28. The method according to claim 18, wherein amplification is done in a total volume of over 22 μl.

29. The method according to claim 18, wherein amplification is done in a total volume of over 25 μl.

30. The method according to claim 18, wherein amplification is done in a total volume between 5 μl and 30 μl.

31. The method according to claim 18, wherein amplification is done in a total volume of between 5 μl and 100 μl.

32. A method of preparing a lyophilized nucleic acid amplification composition comprising:
mixing in liquid form
a. a first reagent mixture for performing a nucleic acid amplification or elongation reaction, and
b. one or more amplification/elongation adjuvants selected from the group consisting of sucrose, sorbitol, methylglucosides, glycine, arginine, proline, erythritol, xylitol, maltose, lactose, FICOLL®, mannitol, dextran, and trehalose; and
lyophilizing the liquid mixture to obtain the lyophilized nucleic acid amplification composition.
